(19) United States
(12) Patent Application Publication
(10) Pub. No.: US 2005/0202081 A1
(43) Pub. Date: Sep. 15, 2005

(54) Stable pharmaceutical compositions comprising ace inhibitor(s)

(76) Inventors: Deepak Bahl, New Delhi (IN); Ravi Kochhar, Haryana (IN); Puneet Sharma, Uttar Pradesh (IN); Vishnubhotla Nagaprasad, Hyderabad (IN); Rajeev Shanker Mathur, New Delhi (IN); Kamal Mehta, Rajasthan (IN)

Correspondence Address: RANBAXY INC., 600 COLLEGE ROAD EAST, SUITE 2100, PRINCETON, NJ, 08540, US

(21) Appl. No.: 10/501,450

(22) PCT Filed: Jan. 14, 2003
(86) PCT No.: PCT/IB03/00063

§ 371 Date: May 18, 2005

(30) Foreign Application Priority Data
Jan. 15, 2002 (IN) 24/DEL/2002

(51) Int. Cl. A61K 31/401; A61K 9/64

(52) U.S. Cl. 424/456; 514/423

(57) ABSTRACT

The invention provides stable pharmaceutical compositions comprising ACE inhibitor(s) and other pharmaceutically active substances which are susceptible to degradation, as well as processes for the preparation thereof, and methods of treatment involving administration of such compositions.

### FIELD OF THE INVENTION

The present invention relates to stable pharmaceutical compositions comprising ace inhibitor(s), which are susceptible to degradation, and processes for the preparation thereof.

### BACKGROUND

Certain Angiotensin Converting Enzyme (ACE) inhibitors, which are useful as antihypertensives, are susceptible to certain types of degradation. ACE inhibitors such as ramipril, quinapril, enalapril, spirapril, lisinopril, benazepril and structurally related drugs can undergo cyclization via internal nucleophilic attack to form substituted diketopiperazines. These drugs can also degrade via hydrolysis (of the side-chain ester group) and oxidation, to form products having unwanted coloration. It has been found that a significant cause of such degradations can be the mechanical stress associated with the manufacturing process of pharmaceutical composition such as compression. The stability of pharmaceutical compositions containing ACE inhibitors can also be negatively influenced by the choice of tabletting auxiliaries. In view of the usefulness of ACE inhibitors in treating hypertension, a number of research endeavors have been directed towards overcoming the inherent instability problem associated with ACE inhibitor-containing compositions.

For example U.S. Pat. Nos. 4,743,450, 4,830,853, 4,793,998, international patent application WO 99/62560, European patent EP 468929 disclose stabilization with various agents. U.S. Pat. Nos. 5,151,433 and 5,442,008 disclose polymeric film-formers as protection against stress, as well as the use of buffers.

Thus, either addition of a stabilizer or a polymeric coat on the active ingredient is believed necessary to stabilize the pharmaceutical composition of ACE inhibitors, which are susceptible to degradation. However, the addition of such stabilizers can produce unwanted pharmacological effects. Coating the active ingredient is quite cumbersome and low yielding moreover it requires specialized equipment.

### SUMMARY

The applicants of the present invention have discovered a process making the use of the above unnecessary. Active ingredient, for example, an ACE inhibitor, which is susceptible to degradation, is applied as a coat to the core, preferably to a compressed core, thereby avoiding degradation (such as cyclization to diketopiperazine) induced by mechanical stress, which builds up during compression. Such an arrangement also avoids the direct contact of the tabletting auxiliaries with the ACE inhibitor, thereby avoiding degradation by any incompatible tablet auxiliaries.

The present invention therefore allows greater flexibility in the choice of tabletting auxiliaries. Moreover, as stabilizers are not required, untoward pharmacological effects, which could occur with the addition of such additives, are nullified. The process can be easily scaled up using the conventional tabletting and coating equipment.

Therefore, the present invention can provide a stable pharmaceutical composition for oral administration of an ACE inhibitor comprising a core coated with a layer of ACE inhibitor(s) and process for preparation thereof.

### DETAILED DESCRIPTION

The core of the present invention is preferably a compressed core, which could be inert or may contain a drug other than the ACE inhibitor susceptible to degradation, such as hydrochlorothiazide, piretanide; and dihydropyridines such as felodipine, nitrendipine, nifedipine, lacidipine or other similar drugs. Alternatively the core may be a sugar or starch particle such as non-pareil sugar seeds, or pregelatinized starch. The core can be of any convenient shape, such as an spheroidal shape. The cores can range from about 25 mg to about 1 gram.

The compressed core may comprise diluent and other formulating agents such as binder, disintegrant, lubricant and glidant. The diluent may be, for example, any pharmaceutically acceptable, non-toxic diluent. Particular examples include lactose, dextrose, sucrose, maltose, microcrystalline cellulose, starch, calcium hydrogen phosphate, mannitol and the like.

Binders may be, for example, starch, sugars, gums, low molecular weight hydroxypropyl methylcellulose, hydroxypropylcellulose or the like. Disintegrant may be, for example, croscarmellose sodium, crospovidone, sodium starch glycolate, bentonite, sodium alginate, hydroxypropylmethylcellulose or the like. Lubricants may be, for example, talc, magnesium stearate, calcium stearate, hydrogenated vegetable oils, stearic acid, sodium stearyl fumarate, sodium benzoate or the like. Glidants may be, for example, colloidal silicon dioxide (aerosil), talc or the like.

The ACE inhibitor layer comprises ACE inhibitor(s), which are susceptible to degradation, including ramipril, spirapril, lisinopril, enalapril, quinapril, benazepril and other structurally related drugs. The process is applicable to other pharmaceutically active agents that are susceptible to mechanical stress-induced or mechanical stress-related degradation. The ACE inhibitor can be micronized, and suspended/dispersed in a solvent to which film forming polymer(s) is added. The film-forming polymer may be, for example, hydroxypropylcellulose, hydroxypropylmethylcellulose, hydroxyethylcellulose, ethylcellulose, cellulose acetate, polyvinylpyrrolidone, gelatin, LustreClear™ (combination of microcrystalline cellulose and carrageenan), combinations of polyvinylalcohol and polyvinylacetate, and the like. The amount of the film forming polymer(s) can be relatively low, to limit the tablet/pellet/beadlet size and the manufacturing effort, but should be sufficient to effectively coat the drug on to the core. The drug to polymer ratio may range from about 1:10 to about 10:1. For example, drug to polymer ratio can be from about 1:2 to about 2:1, or from about 1:1.2 to about 1.2:1, or, for example, about 1:1.

The polymer that is used for binding properties can also protect ACE inhibitors from atmospheric or chemical oxidation and degradation by agents such as atmospheric humidity through, for example, hydrolysis.

The ACE inhibitor layer may optionally contain plasticizers, and is desirably without plasticizers. Stability can be undesirably lessened through the use of plasticizers. Plasticizers which may be excluded from the compositions include polyethyleneglycol, propylene glycol, triethyl citrate, triacetin, dibutylphthalate, diethylphthalate, castor oil, tributyl citrate, glycerol, sorbitol, polysorbates, sorbitan esters and the like. The ACE inhibitor layer may also contain pigments, colorants, antifoaming agents, waxes, monoglycerides, emulsifiers, surfactants or other additives. The layer containing ACE inhibitor or other pharmaceutically active substance can contain such material from about 2% to about 90% by weight of the ACE inhibitor layer, and can also contain film-forming polymer from about 10% to about 98% by weight of the ACE inhibitor layer.

Drug coating solution can be prepared in water, non-aqueous solvents or mixtures thereof. However desirably in water as stability can be lessened through the use of non-aqueous solvents. Solvents that may be excluded are isopropyl alcohol, acetone andmethylene chloride.

A seal coat may optionally separate the core and the ace inhibitor layer to completely seal the tabletting auxiliaries to come in contact with the ACE inhibitor. Similarly, an outer coat may optionally be given on the ACE inhibitor layer to improve the aesthetic appeal of the tablet and to protect it from the atmospheric humidity. The seal coat and outer coat may have the same composition as the ACE inhibitor layer except the drug, or it may have a different composition. For example, the seal coat may contain other polymers, such as Povidone. The seal coat can be prepared from aqueous dispersion of from about 2% to about 30% by weight of film-forming polymer.

The process of the present invention may be carried out in the following manner. The compressed core can be prepared by conventional techniques such as direct compression, wet granulation and dry granulation.

The ACE inhibitor coating dispersion, suspension or solution can be prepared by adding the active ingredient(s) in a solvent with stirring or other mixing. Film forming polymer(s) and other additives can be added to the active ingredient dispersion with stirring or other mixing. The cores are charged into a coating pan and warmed with air to an outlet-air temperature of, for example, about 30° C.-45° C. The ACE inhibitor coating dispersion can be sprayed onto the cores and upon completion, the drug-coated tablets is dried, for example with dry air. Seal coat and outer coat dispersion may be prepared and applied in the similar manner as the ACE inhibitor layer, if required.

The coated tablets after air-drying can be packed into containers impervious to water vapor, e.g. blister packs (alu-alu; PVDC, PE, PVC-alu).

The tablets prepared by the present process may also be filed into capsules. The present process may also be applied to the non-pareil seeds or beadlets, which may then be filled in hard gelatin or starch capsules. Such capsules can have better stability as compared to the conventional capsules.

The compositions disclosed herein may be formulated into solid dosage forms for oral administration, such as, for example, tablets, granules, capsules, pills, and the like. In these cases, the medicaments can be prepared by conventional methods, including a therapeutically effective amount of an ACE inhibitor or other pharmaceutically active substance, and optionally but desirably, pharmaceutically acceptable excipients. In addition to the common dosage forms set out, the compositions may also be administered by controlled release means and/or delivery devices, with modifications known to those of ordinary skill in the art.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

By “therapeutically effective amount” is meant the quantity of a compound or composition according to the invention necessary to prevent, cure or at least partially arrest the symptoms of the disorder and its complications. Amounts effective to achieve this goal will, of course, depend on the severity of the disease and the weight and general state of the patient.

Methods of Treatment

The pharmaceutical compositions provided herein can be utilized for various treatment methods, such as those for treating hypertension, either alone or in combination with thiazide diuretics, as well as for use with stable patients who have demonstrated clinical signs of congestive heart failure within the first few days after sustaining acute myocardial infarction, and also for left ventricular dysfunction and diabetic nephropathy.

The methods include administering to a mammal a therapeutically effective amount of a pharmaceutical composition as described herein.

The administration of pharmaceutical compositions can be by oral or buccal administration. Other methods of administration will be known to those skilled in the art.

Using the process parameters of the present invention, a convenient, reproducible stable pharmaceutical composition of the ACE inhibitors may be obtained. The present invention is further illustrative by, but is by no-means limited to, the following examples.

### EXAMPLES

### Example 1

### Preparation of ACE Inhibitor Formulation

To prepare the tablet cores, in a non-shear blender, microcrystalline cellulose, Pregelatinised starch & Mannitol were mixed and to this mixture sodium stearyl fumarate was added and mixed. The mixture is then compressed to tablets of 100 mg each.

To prepare the seal coating solution, hydroxypropylmethylcellulose, hydroxypropylcellulose, (polyethylene glycol, titanium dioxide, and talc) were dispersed in water with stirring and the suspension homogenized.

To prepare the drug coating suspension, ramipril was dispersed in water with stirring and to it hydroxypropylmethylcellulose, hydroxypropylcellulose, (polyethylene glycol, titanium dioxide, and talc) were added. The suspension was homogenized.

The outer-coating solution was prepared similar to the seal coat solution.

Tablet cores were placed in the coating pan (Hi-Coater) and heated with warm air to an air outlet temperature of about 30° C.-45° C. The seal coating solution was sprayed on the cores. Upon completion the heating was discontinued but the air supply was maintained for about 10 minutes in order to dry the tablets.

The coated cores were sprayed with the drug coating solution and air dried maintaining the process parameters as for the seal coat. Similarly, the outer coating solution was then sprayed on the drug coated cores. The tablets were air dried and extracted from the apparatus and packed in suitable pack. The particular amounts of ingredients for various formulations are tabulated in Tables I, II III, IV and V. Tablets prepared according to the ingredients of these tables were prepared according to the process described above.

TABLE I

| INGREDIENT | WEIGHT(mg) |
|---|---|
| Core | |
| Mannitol | 44.50 |
| Microcrystalline cellulose | 43.50 |
| Pregelatinised starch | 10.0 |
| Sodium Stearyl Fumarate USNF | 2.0 |
| Tablet Weight | 100.00 |
| Seal Coat | 2 |
| Hydroxypropylmethylcellulose(67%) | |
| Hydroxypropylcellulose(6.7%) | |
| Polyethylene glycol(12%) | |
| Titanium dioxide(10%) | |
| Talc(6.3%) | |
| Purified water (q.s) | |
| Target Weight | 102.00 |
| Drug layer | |
| Ramipril | 2.5 |
| Fihn forming Polymer | 2.5 |
| Hydroxypropylmethylcellulose(67%) | |
| Hydroxypropylcellulose(6.7%) | |
| Polyethylene glycol(12%) | |
| Titanium dioxide(10%) | |
| Talc(6.3%) | |
| Purified water (q.s) | |
| Target Weight | 107.00 |
| Outer Coat | 2.0 |
| Hydroxypropylmethylcellulose(67%) | |
| Hydroxypropylcellulose(6.7%) | |
| Polyethylene glycol(12%) | |
| Titanium dioxide(10%) | |
| Talc(6.3%) | |
| Purified water (q.s) | |
| Total Weight | 109.00 |

TABLE II

| INGREDIENT | WEIGHT(MG) |
|---|---|
| Core | |
| Hydrochlorothiazide | 25.0 |
| Mannitol | 40.0 |
| Dibasic Calcium Phosphate (Anhydrous) | 97.855 |
| Starch | 19.60 |
| Pregelatinized Starch | 4.4 |
| Ferric Oxide (Red) | 0.165 |
| Ferric Oxide (Yellow) | 0.33 |
| Purified Water | q.s |
| Pregelatinised Starch | 11.00 |
| Magnesium Stearate | 1.65 |
| Tablet Weight | 200.00 |
| Seal Coat | 4.00 |
| Hydroxypropylmethylcellulose(67%) | |
| Hydroxypropylcellulose(6.7%) | |
| Polyethylene glycol(12%) | |
| Titanium dioxide(10%) | |
| Talc(6.3%) | |
| Purified water (q.s) | |
| Target Weight | 204.00 |
| Drug layer | |
| Ramipril | 5.0 |
| Film forming Polymer | 5.0 |
| Hydroxypropylmethylcellulose(67%) | |
| Hydroxypropylcellulose(6.7%) | |
| Polyethylene glycol(12%) | |
| Titanium dioxide(10%) | |
| Talc(6.3%) | |
| Purified water (q.s) | |
| Target Weight | 214.00 |
| Outer Coat | 4.00 |
| Hydroxypropylmethylcellulose(67%) | |
| Hydroxypropylcellulose(6.7%) | |
| Polyethylene glycol(12%) | |
| Titanium dioxide(10%) | |
| Talc(6.3%) | |
| Purified water (q.s) | |
| Total Weight | 218.00 |

TABLE III

| INGREDIENT | WEIGHT(MG) |
|---|---|
| Core | |
| Mannitol | 44.50 |
| Microcrystalline cellulose | 43.50 |
| Pregelatinised starch | 10.0 |
| Sodium Stearyl Fumarate USNF | 2.0 |
| Tablet Weight | 100.00 |
| Seal Coat | 2 |
| Hydroxypropylmethylcellulose | |
| Purified water (q.s) | |
| Target Weight | 102.00 |
| Drug layer | |
| Ramipril | 2.5 |
| Hydroxypropylmethylcellulose | 2.5 |
| Purified water (q.s) | q.s |
| Target Weight | 107.00 |
| Outer Coat | 2.0 |
| Hydroxypropylmethylcellulose | |
| Purified water (q.s) | |
| Total Weight | 109.00 |

TABLE IV

| INGREDIENT | WEIGHT (MG) |
|---|---|
| Core tablets: | |
| Mannitol | 44.50 |
| Microcrystalline cellulose | 44.50 |
| Pregelatinized starch | 10.0 |
| Sodium Stearyl Fumarate | 1.0 |
| Tablet Weight | 100.0 |
| Pre Coat | |
| Hydroxypropylmethyl cellulose | 2.50 |
| Purified water | q.s |
| Target Weight | 102.0 |
| Drug layer | |
| Ramipril | 3.28 |
| Hydroxypropylmethyl cellulose | 2.98 |
| Hydroxypropyl Cellulose | 0.3 |
| Purified water | q.s |
| Target Weight | 107.25 |

TABLE V

| Ingredients | Qty(mg) |
|---|---|
| Core tablets: | |
| Hydrochlorothiazide | 12.50 |
| Mannitol | 20.0 |
| Dibasic Calcium Phosphate (anhydrous) | 48.928 |
| Maize Starch | 9.80 |
| Pregelatinized Starch | 2.20 |
| Ferric Oxide (Red) | 0.0825 |
| Ferric Oxide (Yellow) | 0.165 |
| Purified Water | q.s |
| Pregelatinised Starch | 5.50 |
| Magnesium Stearate | 0.825 |
| Tablet Weight | 100.00 |
| Pre Coat 1 | |
| Hydroxypropylmethyl cellulose | 1.8125 |
| Hydroxypropyl Cellulose | 0.1875 |
| Titanium Dioxide | 0.50 |
| Purified water | q.s. |
| Target Weight | 102.0 |
| Pre Coat 2 | |
| Hydroxypropylmethyl cellulose | 2.5 |
| Purified water | q.s |
| Target Weight | 104.0 |
| Drug layer | |
| Ramipril | 3.28 |
| Hydroxypropylmethyl cellulose | 2.98 |
| Hydroxypropyl Cellulose | 0.30 |
| Purified water | q.s |
| Target Weight | 109.25 |

Stability comparisons with conventional tablets having ramipril in the core with or without buffer and marketed ramipril tablets (Delix) of Aventis are shown in Table VI below.

TABLE VI
Stability Comparison of 2.5-mg Ramipril Tablets
Prepared by Different Composition/Techniques

| | | Ramipril tablets | | | |
|---|---|---|---|---|---|
| | | | Compressed | Tablets | Delix |
| | | | tablet with tris- | prepared | marketed |
| | | Conventional | (hydroxymethyl) | as per | ramipril |
| Degradation | | compressed | aminomethane | example | tablets of |
| product | Condition | tablet | buffer | 1 | Aventis |
| Diketopiperazine | Initial | 1.91 | 0.048 | 0.262 | |
| (%) | 60° C./2 | 18.48 | 4.61 | 4.42 | 5.91 |
| | weeks | | | | |
| Total Related | Initial | 1.95 | 0.072 | 0.47 | |
| Substances | 60° C./2 | 19.22 | 7.26 | 4.59 | |
| (%) | weeks | | | | |

The stability of tablets prepared according to Tables IV and V was determined at 40° C./75% relative humidity for a period of 6 months. The results of the same are summarized in Table VII and VIII below.

TABLE VII
Stability Data of Ramipril Tablets Prepared as per Table IV
for 6 Months Stored at 40° C./75% Relative Humidity

| | Initial | 1 month | 2 months | 3 months | 6 months |
|---|---|---|---|---|---|
| Diketopiperazine | 0.13 | 1.26 | 1.92 | 2.88 | 4.92 |
| (%) | | | | | |
| Total impurities | 0.16 | 1.48 | 2.06 | 3.19 | 5.51 |
| (%) | | | | | |
| Assay (% w/w) | 103.2 | 101.6 | 99.3 | 98.1 | 95.4 |

TABLE VIII
Stability Data of Ramipril + Hydrochlorothiazide Tablets Prepared
as per Table V for 6 Months Stored at 40° C./75% Relative Humidity

| | Initial | 1 month | 2 months | 3 months | 6 months |
|---|---|---|---|---|---|
| Diketopi- | 0.265 | 1.448 | 2.295 | 3.481 | 6.778 |
| perazine (%) | | | | | |
| Impurities of | 0.407 | 0.436 | 0.531 | 0.701 | 0.829 |
| Hydrochiorothia- | | | | | |
| zide (%) | | | | | |
| Total | 1.289 | 2.573 | 3.094 | 4.338 | 7.159 |
| Impurities (%) | | | | | |
| Assay (% w/w) | | | | | |
| Ramipril Assay | 103.8 | 102.6 | 100.4 | 97.2 | 93.8 |
| (%) | | | | | |
| Hydrochlorothia- | 101.2 | 101.3 | 100.8 | 100.6 | 99.2 |
| zide assay (%) | | | | | |

The data clearly show that the tablets prepared by the processes described herein provide the most stable tablets.

While the present invention has been described in terms of its specific embodiments, certain modifications and equivalents will be apparent to those skilled in the art and are intended to be included within the scope of the present invention.

1. A stable pharmaceutical composition for oral administration of ACE inhibitor(s) comprising
i) a core; coated with a layer of ACE inhibitor(s).
ii) a seal coat on the core; and
iii) an ACE inhibitor layer which is free of plasticizers and organic solvents.

2. The composition of claim 1 wherein the ACE inhibitor is selected from the group consisting of ramipril, quinapril, enalapril, spirapril, lisinopril and benazepril.

3. The composition of claim 1 wherein the layer of ACE inhibitor(s) comprises film forming polymer.

4. The composition of claim 3 wherein the film forming polymer is selected from the group consisting of hydroxypropylcellulose, hydroxypropylmethylcellulose, hydroxyethylcellulose, ethylcellulose, cellulose acetate, polyvinylpyrrolidone, gelatin, a combination of microcrystalline cellulose and carrageenan, and a combination of polyvinyl alcohol and polyvinylacetate.

5. The composition of claim 1, wherein the core is a compressed tablet.

6. The composition of claim 1, wherein the core comprises a sugar sphere or nonpareil seeds.

7. The composition of claim 5 or 6, wherein compressed tablets, sugar spheres or nonpareil seeds are filled into hard gelatin capsules.

8. The composition of claim 1, wherein the core is inert.

9. The composition of claim 1, wherein the core has a pharmaceutically active substance other than the one which is susceptible to degradation by mechanical stress.

10. The composition of claim 9, wherein the pharmaceutically active substance is selected from the group consisting of hydrochlorothiazide, piretanide, and dihydropyridines

11. The composition of claim 1 wherein the seal coat comprises film forming polymer

12. The composition of claim 11 wherein the film forming polymer is selected from the group consisting of hydroxypropylcellulose, hydroxypropylmethylcellulose, hydroxyethylcellulose, ethylcellulose, cellulose acetate, polyvinylpyrrolidone, gelatin, a combination of microcrystalline cellulose and carrageenan, and a combination of polyvinyl alcohol and polyvinylacetate.

13. The composition of claim 1 wherein the ACE inhibitor layer is further surrounded with an outer coat.

14. A process for the preparation of a stable pharmaceutical composition for oral administration of ACE inhibitor(s) comprising applying a seal coat on a core and disposing a layer of an ACE inhibitor dispersion, suspension which is free of plasticizers and organic solvents onto the coated core.

15. The process of claim 14, wherein the layer is disposed as a coating dispersion.

16. The process of claim 15, wherein the coating dispersion is made in aqueous solvent.

17. The process of claim 14, wherein the dispersion is sprayed on the cores.

18. A method of treating a disorder selected from the group consisting of hypertension, congestive heart failure, left ventricular dysfunction and diabetic nephropathy, the method comprising administration of a therapeutically effective amount of the pharmaceutical composition of claim 1 to a patient suffering from such a disorder.
